(19) United States
(12) Patent Application Publication
(10) Pub. No.: US 2026/0053453 A1
(43) Pub. Date: Feb. 26, 2026

(54) PORTABLE X-RAY DEVICE

(71) Applicant: ELEC-FIELD FUTURE CORP., Gyeonggi-do (KR)

(72) Inventor: Han Sung LEE, Gyeonggi-do (KR)

(73) Assignee: ELEC-FIELD FUTURE CORP., Gyeonggi-do (KR)

(21) Appl. No.: 19/103,456

(22) PCT Filed: Aug. 12, 2022
(86) PCT No.: PCT/KR2022/012071

§ 371 Date: Mar. 17, 2025

(51) Int. Cl. A61B 6/00; A61B 6/40; A61B 6/46

(52) U.S. Cl. CPC A61B 6/4405; A61B 6/405; A61B 6/463; A61B 6/465; A61B 6/56

(57) ABSTRACT

The disclosed portable X-ray device according to the present invention comprises: an X-ray unit within which an X-ray source is provided to irradiate a subject with X-rays; a handle unit which protrudes obliquely from the X-ray unit; at least one camera unit which is provided in the X-ray unit to capture an image of the subject; a display unit which displays information captured from the camera unit and X-ray irradiation information of the X-ray unit; and a power supply unit which supplies power to the X-ray unit, the camera unit, and the display unit, wherein the handle unit extends to be inclined downward in the direction away from the X-ray unit. According to such a structure, the ease of gripping the portable X-ray device can be improved and thus the portable X-ray device has excellent ease of use.

### TECHNICAL FIELD

The present disclosure relates to a portable X-ray device, and more particularly, to a portable X-ray device capable of having improved usability by reducing a burden on a user's wrist while gripping it and of increasing efficiency of X-ray irradiation.

### BACKGROUND ART

With the development of industry, portable X-ray devices are becoming increasingly popular that are capable of X-ray irradiation while gripped by users. These portable X-ray devices are highly prevalent in both large hospitals and small hospitals such as dental clinics, making them more accessible to patients who require X-ray imaging for diagnosis of diseases. In addition, the portable X-ray devices have an advantage of reducing the psychological anxiety of patients to have X-rays taken.

Meanwhile, since these portable X-ray devices must be directly gripped and operated by the users, the convenience of user operation is determined by the angle of grip thereof. Accordingly, various studies have been ongoing to improve the convenience of operation of portable X-ray devices in recent years. In addition, there is a growing demand for research to improve the quality of X-ray imaging by guiding even unskilled users to accurately take X-rays.

### DISCLOSURE

### Technical Problem

An object of the present disclosure is to provide a portable X-ray device capable of having improved usability by reducing a burden on a user's wrist while gripping its handle.

Another object of the present disclosure is to provide a portable X-ray device capable of improving alignment and irradiation position accuracy of a subject to be imaged (which is also simply referred to as “subject”) for X-ray irradiation.

### Technical Solution

In accordance with an aspect of the present disclosure, there is provided a portable X-ray device that includes an X-ray unit having an X-ray source provided therein to irradiate a subject with X-rays, a handle unit protruding obliquely from the X-ray unit, at least one camera unit provided in the X-ray unit to capture the subject, a display unit configured to display information captured by the camera unit and X-ray irradiation information from the X-ray unit, and a power supply unit configured to supply power to the X-ray unit, the camera unit, and the display unit, wherein the handle unit extends obliquely downwards in a direction away from the X-ray unit.

The X-ray unit may have a jog dial provided on its rear side.

The display unit may enable signal input by touch.

The power supply unit may include a main battery integrally provided in the handle unit, and an auxiliary battery detachably provided on one side of the X-ray unit. The auxiliary battery may have a larger capacity than the main battery.

The power supply unit may include a charge/discharge board provided within the X-ray unit and electrically connected to the main or auxiliary battery to control charging or discharging of the main or auxiliary battery, and a charging pin electrically connected to the charge/discharge board and provided on one side of the X-ray unit for connection to an external charging source, so as to charge the main battery or the auxiliary battery.

The X-ray unit may include a body provided therein with the X-ray source and a control circuit configured to control X-ray irradiation, a guide tube protruding forwards from the body to guide the irradiation of the subject with X-rays, and a barrier provided between the body and the guide tube to prevent backscattering of X-rays.

The barrier may be integrally provided on the front of the body facing the subject, and the guide tube may be made translucently.

The body and the barrier may be made of a metal material that does not contain lead (Pb), and the barrier may have a thickness corresponding to a lead equivalent of 0.22 t to 0.28 t and a larger width than the body.

The body may be provided therein with a charge/discharge board to control charging and discharging of the power supply unit, and the body may be provided on one side thereof with a charging pin connected to the charge/discharge board and electrically connected to an external charging source, so as to charge the power supply unit.

The camera unit may include a plurality of cameras installed on the respective guide tube and barrier.

The control circuit may exchange signals with the camera unit and the display unit, thereby automatically aligning a position of the subject based on the image captured by the camera unit to display the same on the display unit.

The control circuit may exchange signals with the camera unit and the display unit, thereby automatically adjusting an intensity of X-rays depending on the position of the subject to display the same through the display unit.

The handle unit may extend obliquely downwards from a lower portion of the rear of the body in a direction away from the body.

In accordance with another aspect of the present disclosure, there is provided a portable X-ray device that includes an X-ray unit having an X-ray source provided therein to irradiate a subject with X-rays and made of a metal material that does not contain lead (Pb), a handle unit protruding obliquely from the X-ray unit, at least one camera unit provided in the X-ray unit to capture the subject, a display unit configured to display at least one of information captured by the camera unit and X-ray irradiation information from the X-ray unit, and a power supply unit configured to supply power to the X-ray unit, the camera unit, and the display unit, wherein the handle unit extends obliquely downwards in a direction away from the X-ray unit such that a user puts his/her hand from above the X-ray unit and grips the handle unit.

The X-ray unit may be provided with a jog dial on its rear side, and the display unit may enable signal input by touch.

The power supply unit may include a main battery integrally provided in the handle unit, and an auxiliary battery detachably provided on one side of the X-ray unit. The auxiliary battery may have a larger capacity than the main battery.

The main or auxiliary battery may be electrically connected to an external charging source for charging.

The X-ray unit may include a body provided therein with the X-ray source and a control circuit configured to control X-ray irradiation, a guide tube protruding forwards from the body to guide the irradiation of the subject with X-rays, and a barrier provided between the body and the guide tube to prevent backscattering of X-rays.

The barrier may be integrally provided on the front of the body facing the subject, and the guide tube may be made translucently.

The barrier may have a thickness corresponding to a lead equivalent of 0.22 t to 0.28 t and a larger width than the body.

The body may be provided therein with a charge/discharge board to control charging and discharging of the power supply unit, and the body may be provided on one side thereof with a charging pin connected to the charge/discharge board, so as to charge the power supply unit.

The camera unit may include a plurality of cameras installed on the respective guide tube and barrier.

The control circuit may exchange signals with the camera unit and the display unit, thereby automatically aligning a position of the subject based on the image captured by the camera unit to display the same on the display unit and automatically adjusting an intensity of X-rays depending on the position of the subject to display the same through the display unit.

The handle unit may extend obliquely downwards from a lower portion of the rear of the body in a direction away from the body.

### Advantageous Effects

According to the present disclosure, firstly, a user can grip a handle unit as if gripping a kind of vacuum cleaner since the handle unit is provided to protrude from an X-ray unit so that the user puts his/her hand from above and grips the handle unit. As a result, it is possible to provide excellent ease of use for a long time by reducing the burden on a user's wrist and improving a degree of wrist freedom.

Secondly, it is possible to improve the safety of use for the user by providing a barrier between a body and a guide tube of the X-ray unit to prevent backscattering of X-rays. In particular, it is possible to provide a portable X-ray device with excellent durability since the barrier is made of a metal material that does not contain lead together with the body.

Thirdly, by application of vision systems that allow the user to check images captured by a camera unit, an existing thick translucent shielding means containing lead is unnecessary. As a result, it is relatively more advantageous in terms of improving safety of use compared to existing devices.

Fourthly, the position of a subject can be automatically aligned and X-ray conditions can be automatically set up based on the captured image of the subject, which can be provided to the user through a display unit. Therefore, it offers excellent alignment and operability even for unskilled users.

Fifthly, X-ray irradiation can be easily made through a touch-type display unit and a jog dial.

Sixthly, it is possible to increase portability due to the lightweight body since an auxiliary battery with a relatively larger capacity than a main battery is be optionally mounted together with the main battery.

### BRIEF DESCRIPTION OF DRAWINGS

FIG. 1 is a view schematically illustrating a portable X-ray device according to a preferred embodiment of the present disclosure.

FIG. 2 is a top view schematically illustrating the portable X-ray device of FIG. 1 according to the embodiment of the present disclosure.

FIG. 3 is a view schematically illustrating a state in which the portable X-ray device of FIG. 1 is placed on a charging stand according to the embodiment of the present disclosure.

FIG. 4 is an image schematically illustrating information displayed on the display panel of FIG. 1.

FIG. 5 is an image schematically illustrating an example of modification of the information displayed on the display panel of FIG. 1.

FIG. 6 is an image schematically illustrating another example of modification of the information displayed on the display panel of FIG. 1.

### MODE FOR DISCLOSURE

Hereinafter, exemplary embodiments of the present disclosure will be described with reference to the accompanying drawings. It will be apparent to those skilled in the art that the present disclosure is not limited to such embodiments, various variations and modifications may be made by adding, changing, or removing components without departing from the spirit and scope of the disclosure, and these variations and modifications fall within the spirit and scope of the disclosure.

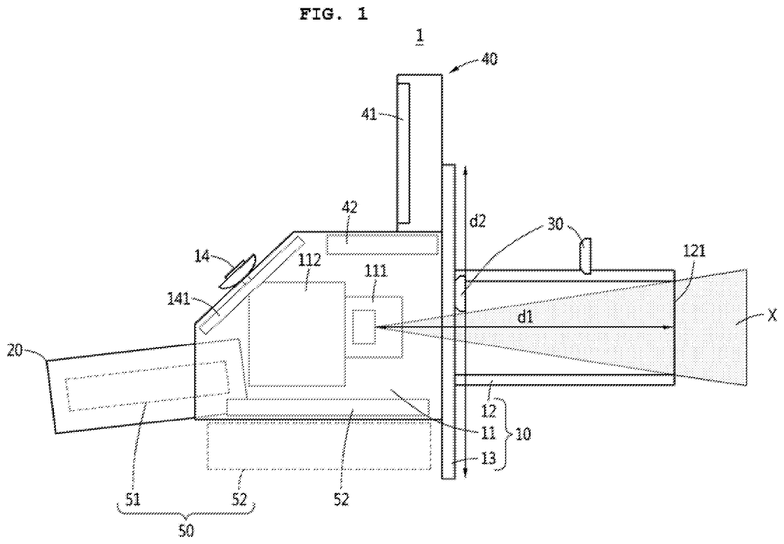

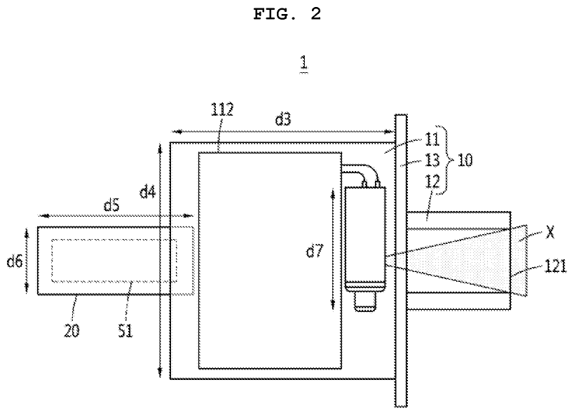

Referring to FIGS. 1 and 2, a portable X-ray device 1 according to a preferred embodiment of the present disclosure includes an X-ray unit 10, a handle unit 20, a camera unit 30, a display unit 40, and a power supply unit 50.

For reference, the portable X-ray device 1 described in the present disclosure is exemplified as a portable X-ray imaging device for taking a low-dose X-ray of a patient's affected area for the purpose of treatment such as in dental clinics. It is, however, obvious that the portable X-ray device 1 may be used for various purposes, such as in industrial facilities, rather than for the purpose of treatment.

The X-ray unit 10 irradiates a subject (not shown) with X-rays X. The X-ray unit 10 includes a body 11, a guide tube 12, and a barrier 13.

The body 11 has a space for accommodating a predetermined component therein, and an X-ray source 111 and a control circuit 112 are accommodated in the space within the body 11. The body 11 is made of a metal material excluding lead (Pb), and in this embodiment, may include a metal frame.

For reference, an existing X-ray device has a body that is typically made of a translucent material containing lead. However, in this embodiment, by application of vision systems such as the camera unit 30 and the display unit 40 to be described in detail later, it is unnecessary for the body to be made of a translucent material containing lead to visually identify an existing subject.

The X-ray source 111 is intended to generate X-rays X and may include a general shieldable X-ray tube. Since the X-ray source 111 including the X-ray tube and the like is not a key component of the present disclosure, a detailed description and illustration thereof will be omitted.

The control circuit 112 is connected to the X-ray source 111 to control the generation of X-rays X by the X-ray source 111. In addition, the control circuit 112 may be electrically connected to the camera unit 30, the display unit 40, and the power supply unit 50, which will be described in detail later, so as to generally control X-ray imaging by the portable X-ray device 1. For reference, the control circuit 112 may be provided as a high voltage multiplier circuit.

The guide tube 12 protrudes forwards from the body 11 to guide the irradiation of the subject with X-rays X. For this purpose, the guide tube 12 is provided to face the X-ray source 111 and protrudes outwards from the body 11 toward the subject. The length, diameter, and the like of the guide tube 12 are not limited to the example illustrated. For reference, the guide tube 12 may be made of the same material as the body 11, or more preferably, may be made translucently. In other words, the existing X-ray device has an opaque guide tube to guide X-ray irradiation, but the guide tube 12 in this embodiment may be made translucently in order to further enhance user identification.

Meanwhile, the guide tube 12 has an irradiation hole 121 formed at the end thereof to irradiate the subject with X-rays X therethrough. In this case, the distance d1 between the X-ray source 111 and the irradiation hole 121 of the guide tube 12 may be, but not limited to, approximately 200 mm.

The barrier 13 is provided between the body 11 and the guide tube 12 to prevent backscattering of X-rays X. The barrier 13 is made of a metal material that does not contain lead (Pb), just like the body 11, and is more preferably provided integrally on the front of the body 11 facing the subject. Here, the barrier 13 is made of a metal material that does not contain lead (Pb), which is one of shielding materials, and has a thickness corresponding to a lead (Pb) equivalent of 0.25 t. As a result, the barrier 13 may prevent the X-rays X irradiated to capture the subject from interfering with a user due to backscattering, thereby contributing to an improvement in safety of use.

As illustrated in FIGS. 1 and 2 it is more preferable that the barrier 13 blocks x-rays X from entering the body 11 due to backscattering by having a width that is larger than the width of the body 11 and the guide tube 12. For reference, although the width d2 of the barrier 13 is exemplified as being approximately 160 mm in this embodiment, the present disclosure is not limited thereto.

For reference, as illustrated in FIG. 2, the body 11 has a rectangular parallelepiped shape with a length d3 of approximately 160 mm and a width d4 of approximately 140 mm. In addition, the handle unit 20 protruding rearwards from the body 11 may be set to have a length d5 of approximately 100 mm and a width d6 of approximately 40 mm. Moreover, the X-ray source 111 provided within the body 11 may have a length d7 of approximately 85 mm. The dimensions d1 to d7 of the portable X-ray device 1 are merely examples and are not limited.

Meanwhile, the X-ray unit 10 has a jog dial 14 provided on the rear side thereof. The application of the jog dial 14 allows the portable X-ray device 1 to be used with simple operation, which can improve the convenience of user operation together with the display unit 40 to be described in detail later. In this case, the jog dial 14 may be electrically connected to an operation control board 141 connected to the control circuit 112 inside the body 11, so as to input operation signals to the control circuit 112 provided within the body 11 by operating the jog dial 14.

The handle unit 20 is provided to protrude obliquely from the rear side of the X-ray unit 10. More specifically, the handle unit 20 is provided to protrude obliquely from the X-ray unit 10 such that the user puts his/her hand from above the X-ray unit 10 and grips the handle unit 20. In other words, the handle unit 20 is positioned at the lower portion of the rear facing the front of the X-ray unit 10 and is inclined downwards in a direction away from the body 11. As a result, the user may put his/her hand from above and grip the handle unit 20, as if gripping a kind of vacuum cleaner.

In summary, the handle unit 20 may be designed with a high degree of wrist freedom in order to reduce the burden on the wrist of the user who grips the handle unit 20. As a result, it is possible to improve usability by reducing the load on the user's wrist even if the portable X-ray device 1 is used for a long time.

The camera unit 30 is provided in the X-ray unit 10 to capture the subject. The camera unit 30 may consist of a plurality of camera units arranged at different positions to capture the subject. In this embodiment, the camera unit 30 is exemplified as consisting of a total of two camera units disposed, one each, on the guide tube 12 and barrier 13 of the X-ray unit 10, as illustrated in FIG. 1. It is, however, obvious that the installation location, number, and the like of the camera units 30 are not limited to the example illustrated.

The display unit 40 displays information captured by the camera unit 30 and information from the X-ray unit 10. The display unit 40 includes a display panel 41 and a display control board 42.

The display panel 41 displays an image or video (hereinafter, collectively referred to as “image”) captured by the camera unit 30. As a result, the user may conduct irradiation of the subject with X-rays X while directly checking the image displayed on the display panel 41 with the naked eye. In addition, the display panel 41 is provided to enable input of signals by touch thereon, which may allow operation signals to be input together with the jog dial 14.

The display control board 42 is provided inside the body 11 of the X-ray unit 10 so as to be electrically connected to the display panel 41. Here, the display control board 42 may be electrically connected to the control circuit 112 of the X-ray unit 10, so as to control general control signals for X-ray irradiation from the X-ray unit 10 to be displayed through the display panel 41.

Meanwhile, when the camera unit 30 captures the subject in real time and provides it to the display unit 40 to be described in detail later, the camera unit 30 may automatically align the position of the subject for X-ray irradiation without the user directly identifying that subject with the naked eye. More specifically, the control circuit 112 of the X-ray unit 10 has a built-in sensor (not shown), and the camera unit 30 automatically aligns the position of X-ray imaging with the subject through signal exchange with the sensor (not shown) of the control circuit 112. In addition, the control circuit 112 may control the intensity of X-rays X to be automatically adjusted depending on the position of the subject based on the image captured by the camera unit 30 through signal exchange with the display unit 40 and the camera unit 30, and may control the intensity of X-rays X to be displayed on the display unit 40.

As such, since the image captured by the camera unit 30 is displayed through the display unit 40 even if the user does not directly identify the subject with the naked eye, the alignment the subject for X-ray irradiation is possible even if the body 11 and barrier 13 of the X-ray unit 10 are made of an opaque metal material. The information displayed through the display unit 40 will be described in more detail later along with the description of the operation of the portable X-ray device 1.

The power supply unit 50 supplies power to the X-ray unit 10, the camera unit 30, and the display unit 40. The power supply unit 50 includes a main battery 51 and an auxiliary battery 52.

As illustrated in FIGS. 1 and 2, the main battery 51 is provided integrally with the handle unit 20 to supply power for the overall operation of the portable X-ray device 1. The auxiliary battery 52 is detachably provided on one side of the X-ray unit 10 to assist the power supply of the main battery 51. Here, the main battery 51 may also be detachably provided on the handle unit 20, just like the auxiliary battery 52.

Meanwhile, the auxiliary battery 52 may have a relatively larger capacity than the main battery 51. As a result, the main battery 51 is relatively lighter in weight because of having a smaller capacity than the auxiliary battery 52. As such, the main battery 51 is provided to have a smaller capacity so as to be lighter in weight than the auxiliary battery 52, which is advantageous in improving the portability and usability of the portable X-ray device 1.

The main and auxiliary batteries 51 and 52 may be placed on a charging stand 54 for charging. For this purpose, the X-ray unit 10 is provided with a first charging pin 531 on one side thereof, more preferably on the bottom of the body 11 thereof, wherein the first charging pin 531 is electrically connected to the charging stand 54 which is an external charging source. Here, the first charging pin 531 is electrically connected to a second charging pin 541 provided on the charging stand 54, thereby charging the main or auxiliary battery 51 or 52 with external power.

In this case, the body 11 of the X-ray unit 10 is provided therein with a charge/discharge board 53, which is electrically connected to the first charging pin 531 and electrically controls the main and auxiliary batteries 51 and 52. Here, the charge/discharge board 53 may be electrically connected to the control circuit 112, which allows a state of charge to be displayed through the display unit 40 electrically connected thereto. For reference, the charging stand 54 may be powered through a charging adapter 55.

The X-ray operation of the portable X-ray device 1 according to the present disclosure having the above configuration will be described with reference to FIGS. 1 to 3.

As illustrated in FIG. 1, the user grips the handle unit 20 such that the subject faces the guide tube 12 of the X-ray unit 10. In this case, the user does not grip the handle unit 20 like an existing type of gun, but puts his/her hand from above the handle unit 20 and grips it. In other words, the user grips the handle unit 20, similar to gripping a typical vacuum cleaner, thereby reducing wrist strain such as wrist bending and providing an excellent degree of wrist freedom.

The user then inputs an X-ray generation signal by operating the jog dial 14 provided on one side of the X-ray unit 10 or the display panel 41 capable of signal input by touch. In this case, the user receives the image captured by the camera unit 30 through the display panel 41 and aligns the subject for X-ray irradiation based thereon.

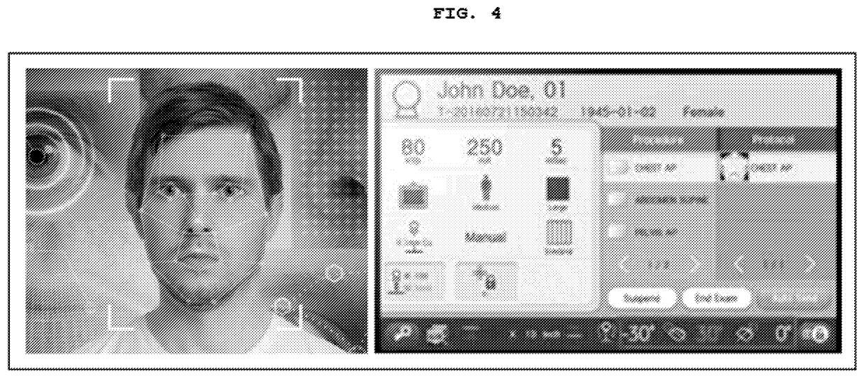

For reference, the camera unit 30 automatically sets up X-ray imaging by capturing the subject and automatically aligning the subject through signal exchange with the sensor accommodated in the control circuit 112. In this case, the image captured by the camera unit 30 is provided to the user through the display panel 41 of the display unit 40, as illustrated in FIG. 4.

Meanwhile, as illustrated in FIG. 4, the information displayed through the display panel 41 of the display unit 40 may include information for X-ray imaging together with the image of the subject. In this case, the control circuit 112 of the X-ray unit 10 exchanges signals with the camera unit 30 and the display unit 40, which allows the intensity and irradiation time of X-rays X to be automatically set up and displayed on the display panel 41, along with the automatic position alignment of the subject for X-ray irradiation.

Here, in this embodiment, by application of vision systems in which the image captured by the camera unit 30 is displayed on the display unit 40, it is unnecessary for the body 11 and the barrier 13 to be made of a translucent material containing lead (Pb) as in an existing X-ray device. As a result, it is possible to ensure the field of view of the user and effectively prevent backscattering of X-rays X even if the barrier 13 including the metal frame is applied between the guide tube 12 and the body 11 for X-ray irradiation.

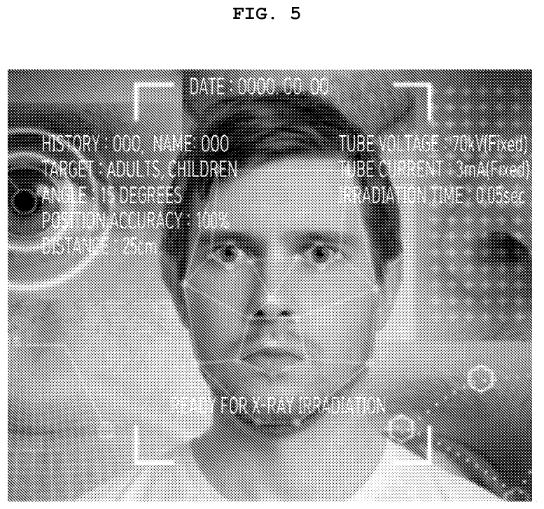

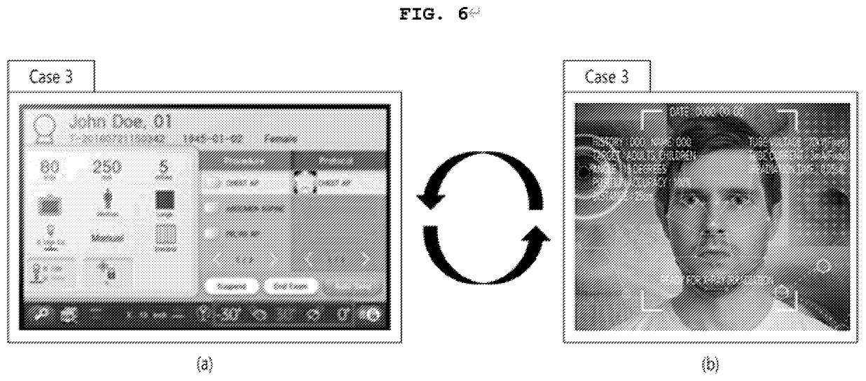

For reference, the information displayed through the display panel 41 of the display unit 40 is not limited to the example of FIG. 4, and an example of modification is also possible in which a captured subject image, X-ray alignment information, X-ray irradiation information including the intensity of X-rays X are simultaneously displayed as one image as in illustrated FIG. 5. In addition, another example of modification is also possible in which X-ray X irradiation information such as in FIG. 6(a) or a captured subject image such as in FIG. 6(b) are switched and displayed on the display panel 41 depending on the situation.

When the X-ray irradiation position alignment, irradiation conditions, and the like for the subject are automatically set up, the user inputs an X-ray X generation signal by operating the jog dial 14 or the touch-type display panel 41. As a result, the X-ray source 111 generates X-rays X and ultimately irradiates the subject with the X-rays X. Here, during X-ray irradiation, power is supplied through the main battery 51 of the power supply unit 50. Alternatively, if the power supply from the main battery 51 is insufficient, power may be supplied through the additionally mounted auxiliary battery 52.

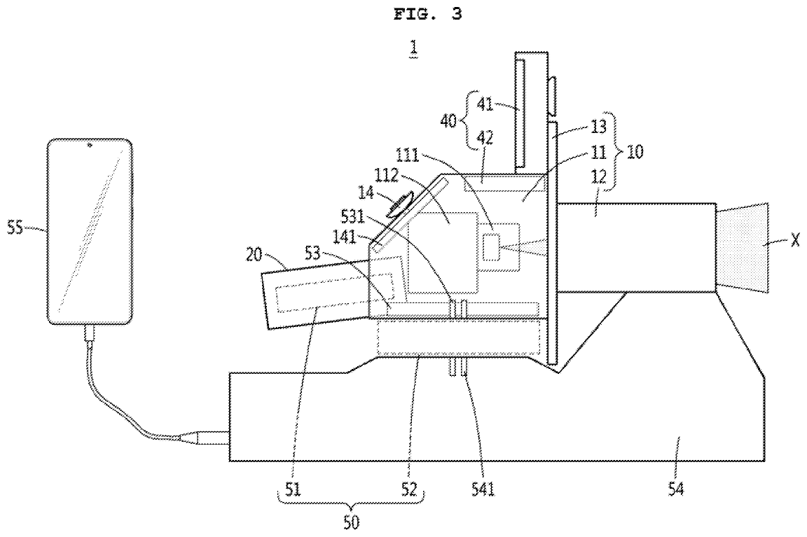

When the X-ray X irradiation of the portable X-ray device 1 is completed, the first charging pin 531 provided on the bottom of the body 11 is placed and connected to the second charging pin 541 of the charging stand 54 as illustrated in FIG. 3. As a result, the main or auxiliary battery 51 or 52 is placed and charged on the charging stand 54.

Although the present disclosure has been described with respect to the preferred embodiments, it will be understood by those skilled in the art that various modifications and variations can be made without departing from the spirit and scope of the disclosure as defined in the appended claims.

1. A portable X-ray device comprising:
an X-ray unit having an X-ray source provided therein to irradiate a subject with X-rays;
a handle unit protruding obliquely from the X-ray unit;
at least one camera unit provided in the X-ray unit to capture the subject;
a display unit configured to display information captured by the camera unit and X-ray irradiation information from the X-ray unit; and
a power supply unit configured to supply power to the X-ray unit, the camera unit, and the display unit,
wherein the handle unit extends obliquely downwards in a direction away from the X-ray unit.

2. (canceled)

3. (canceled)

4. The portable X-ray device according to claim 1, wherein the power supply unit comprises:
a main battery integrally provided in the handle unit; and
an auxiliary battery detachably provided on one side of the X-ray unit, and
wherein the auxiliary battery has a larger capacity than the main battery.

5. The portable X-ray device according to claim 4, wherein the power supply unit comprises a charge/discharge board provided within the X-ray unit and electrically connected to the main or auxiliary battery to control charging or discharging of the main or auxiliary battery, and a charging pin electrically connected to the charge/discharge board and provided on one side of the X-ray unit for connection to an external charging source, so as to charge the main battery or the auxiliary battery.

6. The portable X-ray device according to claim 1, wherein the X-ray unit comprises:
a body provided therein with the X-ray source and a control circuit configured to control X-ray irradiation;
a guide tube protruding forwards from the body to guide the irradiation of the subject with X-rays; and
a barrier provided between the body and the guide tube to prevent backscattering of X-rays.

7. The portable X-ray device according to claim 6, wherein:
the barrier is integrally provided on the front of the body facing the subject; and
the guide tube is made translucently.

8. The portable X-ray device according to claim 6, wherein:
the body and the barrier are made of a metal material that does not contain lead (Pb); and
the barrier has a thickness corresponding to a lead equivalent of 0.22 t to 0.28 t and a larger width than the body.

9. The portable X-ray device according to claim 6, wherein the body is provided therein with a charge/discharge board to control charging and discharging of the power supply unit, and the body is provided on one side thereof with a charging pin connected to the charge/discharge board and electrically connected to an external charging source, so as to charge the power supply unit.

10. The portable X-ray device according to claim 6, wherein the camera unit comprises a plurality of cameras installed on the respective guide tube and barrier.

11. The portable X-ray device according to claim 6, wherein the control circuit exchanges signals with the camera unit and the display unit, thereby automatically aligning a position of the subject based on the image captured by the camera unit to display the same on the display unit.

12. The portable X-ray device according to claim 6, wherein the control circuit exchanges signals with the camera unit and the display unit, thereby automatically adjusting an intensity of X-rays depending on the position of the subject to display the same through the display unit.

13. The portable X-ray device according to claim 6, wherein the handle unit extends obliquely downwards from a lower portion of the rear of the body in a direction away from the body.

14. A portable X-ray device comprising:
an X-ray unit having an X-ray source provided therein to irradiate a subject with X-rays and made of a metal material that does not contain lead (Pb);
a handle unit protruding obliquely from the X-ray unit;
at least one camera unit provided in the X-ray unit to capture the subject;
a display unit configured to display at least one of information captured by the camera unit and X-ray irradiation information from the X-ray unit; and
a power supply unit configured to supply power to the X-ray unit, the camera unit, and the display unit,
wherein the handle unit extends obliquely downwards in a direction away from the X-ray unit such that a user puts his/her hand from above the X-ray unit and grips the handle unit.

15. (canceled)

16. The portable X-ray device according to claim 14, wherein the power supply unit comprises:
a main battery provided integrally in the handle unit; and
an auxiliary battery detachably provided on one side of the X-ray unit, and
wherein the auxiliary battery has a larger capacity than the main battery.

17. (canceled)

18. The portable X-ray device according to claim 14, wherein the X-ray unit comprises:
a body provided therein with the X-ray source and a control circuit configured to control X-ray irradiation;
a guide tube protruding forwards from the body to guide the irradiation of the subject with X-rays; and
a barrier provided between the body and the guide tube to prevent backscattering of X-rays.

19. The portable X-ray device according to claim 18, wherein:
the barrier is integrally provided on the front of the body facing the subject; and
the guide tube is made translucently.

20. The portable X-ray device according to claim 18, wherein the barrier has a thickness corresponding to a lead equivalent of 0.22 t to 0.28 t and a larger width than the body.

21. The portable X-ray device according to claim 18, wherein the body is provided therein with a charge/discharge board to control charging and discharging of the power supply unit, and the body is provided on one side thereof with a charging pin connected to the charge/discharge board, so as to charge the power supply unit.

22. The portable X-ray device according to claim 18, wherein the camera unit comprises a plurality of cameras installed on the respective guide tube and barrier.

23. The portable X-ray device according to claim 18, wherein the control circuit exchanges signals with the camera unit and the display unit, thereby automatically aligning a position of the subject based on the image captured by the camera unit to display the same on the display unit and automatically adjusting an intensity of X-rays depending on the position of the subject to display the same through the display unit.

24. The portable X-ray device according to claim 18, wherein the handle unit extends obliquely downwards from a lower portion of the rear of the body in a direction away from the body.
